(19) United States
(12) Patent Application Publication
(10) Pub. No.: US 2022/0110661 A1
(43) Pub. Date: Apr. 14, 2022

(54) SCOLIOSIS CORRECTION SYSTEMS, METHODS, AND INSTRUMENTS

(71) Applicant: GLOBUS MEDICAL, INC., Audubon, PA (US)

(72) Inventor: John LaColla, West Chester, PA (US)

(21) Appl. No.: 17/482,693

(22) Filed: Sep. 23, 2021

Related U.S. Application Data
(60) Provisional application No. 63/090,501, filed on Oct. 12, 2020.

(51) Int. Cl. A61B 17/70; A61B 17/80

(52) U.S. Cl. CPC A61B 17/7022; A61B 17/7049; A61B 17/7032; A61B 2017/564; A61B 17/809; A61B 17/7043; A61B 17/7058

(57) ABSTRACT

Implants, systems, and methods for securing multiple cords to bone. The fixation system may be used for correcting scoliosis with a fusionless double corded device. The system may include first and second cords, a single bone fastener, one or more tulip structures configured for receiving one or both of the first and second cords, and one or more locking caps adapted to lock one or both of the first and second cords.

### CROSS-REFERENCE TO RELATED APPLICATIONS

This application is a non-provisional of U.S. Provisional Patent Application No. 63/090,501, filed Oct. 12, 2020, which is incorporated by reference herein in its entirety for all purposes.

This application is also related to U.S. patent application Ser. No. 16/284,394, filed Feb. 25, 2019, which is incorporated by reference herein in its entirety for all purposes.

### FIELD

The present disclosure provides devices, systems, and instruments for use in spinal surgeries.

### BACKGROUND

The vertebrate spine is the axis of the skeleton providing structural support for the other parts of the body. Scoliosis is a term used to describe any abnormal, sideway curvature of the spine. The most common form of scoliosis for patients between the age of 10 and 18 years is termed adolescent idiopathic scoliosis (AIS). Although the particular cause of this type of scoliosis is still unknown, advancements in the medical field have enabled doctors to increase the likelihood of successfully treating scoliosis.

One type of spinal surgery for treating scoliosis is the use of implantable rods. One or more rods are implanted into the patient through the back of the spine. The rods may be secured to the spine to correct the condition. There remains a need for improved systems that facilitate scoliosis correction and serve as a means for deformity correction with or without fusion.

### SUMMARY

To meet this and other needs, scoliosis correction devices, systems, methods, and instruments are provided. The systems offer implants and instruments for deformity correction with or without fusion. Traditional rods or a flexible, durable, biocompatible cord may be secured to the spine. The system may apply fixation on the convexity of the scoliotic vertebrae to limit growth on the convex side and allow unilateral growth on the concave side. The system may include a double cord construct in which two cords are installed via a single bone fastener. The second cord may provide additional rotational stability as well as long term dynamic performance. The system creates an optimized construct for spinal alignment to maintain stability while allowing growth in skeletally immature patients. In addition, the instruments facilitate rod or cord insertion and/or tensioning, for example, using an anterior or posterior approach.

According to one embodiment, a spinal system includes a fastener system comprising a fastener, a staple, and a locking cap. The fastener has a threaded portion and a head portion configured to move relative to the threaded portion. The staple includes a ring configured to surround the threaded portion of the fastener and a plurality of prongs configured to engage bone. A rod or cord may be provided in the system and is configured to be retained within one or more of the fastener systems. The rod or cord may be tensioned to impart a compressive force to the scoliotic vertebrae.

According to another embodiment, a spinal system includes a first fastener system, a second fastener system, and a cord. The first fastener system includes a first fastener, a first staple, and a first locking cap. The first fastener has a first threaded portion and a first head portion configured to move relative to the first threaded portion. The first staple includes a first ring configured to surround the first threaded portion of the first fastener and a first plurality of prongs configured to engage bone. The second fastener system includes a second fastener, a second staple, and a second locking cap. The second fastener has a second threaded portion and a second head portion configured to move relative to the second threaded portion. The second staple includes a second ring configured to surround the second threaded portion of the second fastener and a second plurality of prongs configured to engage bone. The cord extends between at least the first fastener system and the second fastener system. The cord is tensioned between the first fastener system and the second fastener system, for example, to correct a curve of the spine.

According to another embodiment, an instrument for tensioning the cord includes a body having an opening. The opening receives a compressor tube, and the compressor tube receives a threaded shaft therethrough. The cord is configured to be affixed to a distal end of the threaded shaft. Movement of the threaded shaft applies a compressive force to the cord to tension the cord within the fastener system.

According to yet another embodiment, an instrument for tensioning the cord may include a first portion and a second portion coupled to the first portion at a pivot point, thereby pivotally connecting the first and second portions together. The first portion includes a first tubular member and the second portion includes a second tubular member. The first and second tubular members are aligned along a common axis such that the opening is a central longitudinal opening extending through the first and second tubular members. In a second position, the first and second tubular members are not aligned along the common axis and are angled relative to one another. The first portion includes a first handle portion extending from the first tubular member and the second portion includes a second handle portion extending from the second tubular member. Distal ends of the first and second handle portions may be connected with a ratchet. The ratchet may extend from the distal end of the second handle portion at a pivot, and the ratchet is engageable with a pawl on the first handle portion. The ratchet may include a plurality of uni-directional teeth. The instrument may include a force gauge and a plurality of indicia, and when the force gauge moves, the plurality of indicia indicate the amount of compressive force on the cord and provided by the instrument.

According to yet another embodiment, an instrument for tensioning the cord includes a first, main handle that grasps the compressor tube and the threaded shaft. The instrument may include a second handle (e.g., a T-handle) or a knob. When using two handles, the two handles may be pulled apart from one another to impart the compressive forces on the cord. As an alternative to the T-handle, a knob may be used. The knob may have an internal thread to threadedly engage the threaded shaft. The knob may be turned to apply the compressive force to the cord.

According to yet another embodiment, a method of installing the system may include inserting one or more staples into an anterior aspect of each vertebral body. After the staple is fully seated against the vertebral body, the fastener may be inserted through the opening in the staple. Once the fasteners are placed, the cord may be selected and positioned within the heads of each fastener. The non-threaded locking caps may be installed in the heads of the fasteners. The cord is tensioned, for example, with the one or more instruments described herein, and after final tensioning of the cord, the locking caps are fully tightened to maintain the tension on the cord.

According to one embodiment, a dual cord fixation system for correcting scoliosis includes first and second cords, a base, a tulip structure disposed on the base for receiving at least one of the first and second cords, and a locking cap disposed in the tulip over at least one of the first and second cords and adapted to lock at least one of the first and second cords.

The dual cord fixation system may include one or more of the following features. The tulip structure may include a body with a pair of upwardly extending arms which define a U-shaped channel therebetween sized and shaped to receive at least one of the first and second cords. The base may include a plate having an upper surface and an opposed lower surface. The tulip structure may be integrated with the upper surface of the plate. The plate may be curved with the lower surface having a concave surface. The plate may include one or more prongs extending downwardly from the lower surface of the plate. The system may include a second tulip structure integral with the upper surface of the plate such that each tulip structure retains one of the first and second cords. The plate may define an opening extending therethrough, and a fastener may be receivable through the opening in the plate. The fastener may include a head and a threaded shaft portion configured to engage bone such that the head may be receivable in the opening through the plate. The locking cap may include a non-threaded outer portion and a threaded inner portion receivable through the outer portion such that threading the inner portion downwardly secures at least one of the first and second cords in the tulip structure.

According to another embodiment, a multiple cord fixation system for correcting scoliosis may include a single bone fastener including a threaded shaft portion configured to engage bone, first and second tulips each including a body with a pair of upwardly extending arms defining a U-shaped channel therebetween, and a plurality of cords configured to be secured in the first and second tulips. The system may be configured to secure the plurality of cords to the single bone fastener, thereby reducing the number of vertebral body screws required during the procedure.

The multiple cord fixation system may include one or more of the following features. The system may include first and second locking caps disposed in the first and second tulips over the cords and adapted to lock the cords therein. In one embodiment, the first and second tulips may be affixed to a plate. The fastener may be receivable through a central opening located between the first and second tulips. The plate may include a pair of prongs extending downwardly and the prongs may be positioned beneath each of the first and second tulips. In another embodiment, one of the first and second tulips may be attached to a plate, and the other of the first and second tulips may be integral with the bone fastener. The fastener may be receivable through a laterally offset opening through the plate, and the tulip attached to the bone fastener may be positionable above the plate.

According to another embodiment, a method of correcting scoliosis of the spine may include one or more of the following steps: (1) positioning a plate against a vertebra, the plate including a first tulip disposed on the plate for receiving a first cord and a second tulip disposed on the plate for receiving a second cord; (2) placing a single fastener through an opening in the plate and securing the fastener to the vertebra; (3) positioning a first cord through the first tulip and securing the first cord therein with a first locking cap; and (4) positioning a second cord through the second tulip and securing the second cord therein with a second locking cap, wherein the first and second cords are affixed to additional fastener systems on adjacent vertebrae to thereby stabilize the spine. The method may further include tensioning the first and second cords to correct a curvature of the spine. The first and second cords may be aligned in parallel to one another.

According to another embodiment, a single head fastener system for correcting scoliosis includes first and second cords, a bone fastener having a single head tulip structure for receiving both of the first and second cords, and a locking cap disposed in the tulip over both of the first and second cords and adapted to lock both of the first and second cords.

The single head fastener system may include one or more of the following features. The tulip structure may include a body with a pair of upwardly extending arms that define a U-shaped channel therebetween sized and shaped to receive both of the first and second cords. The U-shaped channel may be sized to receive both of the first and second cords arranged vertically stacked on top of one another. The first and second cords may be layered on top of each other in direct contact with one another. The system may further include an intermediate locking washer positionable in the tulip structure between the first and second cords. The locking washer may have a ring-shaped body with a central recess for engagement with an instrument. The U-shaped channel may be sized to receive both of the first and second cords arranged laterally side by side. In one embodiment, the locking cap may include a non-threaded outer portion and a threaded inner portion receivable through the outer portion such that threading the inner portion downwardly secures both of the first and second cords in the tulip structure. In another embodiment, the locking cap may include a non-threaded outer portion and a pair of threaded inner portions each receivable through the outer portion such that threading each of the inner portions downwardly independently secures the first and second cords in the tulip structure. The fastener may include a threaded shaft portion configured to engage bone and the tulip structure may be integral with the threaded shaft portion of the fastener.

According to another embodiment, a fastener system for correcting scoliosis includes a single bone fastener including a threaded shaft portion configured to engage bone, a single tulip including a body with a pair of upwardly extending arms defining a U-shaped channel therebetween, and a plurality of cords configured to be secured in the single tulip, wherein the system secures the plurality of cords to the single bone fastener.

The fastener system may include one or more of the following features. The plurality of cords may be arranged vertically stacked on top of one another in the tulip. The plurality of cords may be arranged laterally side by side in the tulip. The system may further include a single locking cap disposed in the tulip over the cords and adapted to lock the plurality of cords therein. The locking cap may include a non-threaded outer portion and a threaded inner portion receivable through the outer portion such that threading the inner portion downwardly secures all of the cords in the tulip. The locking cap may include a non-threaded outer portion and a pair of threaded inner portions each receivable through the outer portion such that threading each of the inner portions downwardly independently secures the cords in the tulip.

According to another embodiment, a method of correcting scoliosis of the spine may include one or more of the following steps: (1) securing a single bone fastener to a vertebra, the fastener including a single tulip including a body with a pair of upwardly extending arms defining a U-shaped channel therebetween; (2) positioning first and second cords into the U-shaped of the tulip; and (3) securing the first and second cords in the tulip with a locking cap, wherein the first and second cords are affixed to additional fastener systems on adjacent vertebrae to thereby stabilize the spine. The first and second cords may be aligned in parallel. The first and second cords may be arranged vertically stacked on top of one another in the tulip or the first and second cords may be arranged laterally side by side in the tulip.

According to yet another embodiment, a kit includes one or more of the components, implants, instruments, or systems described herein. For example, the kit may include a plurality of fastener systems, for example, of different sizes, cords and/or rods, and one or more instruments including instruments for inserting and/or tensioning the cord(s).

### BRIEF DESCRIPTION OF THE DRAWINGS

A more complete understanding of the present disclosure, and the attendant advantages and features thereof, will be more readily understood by reference to the following detailed description when considered in conjunction with the accompanying drawings wherein:

FIG. 1 is a perspective view of a spine with a spinal system according to one embodiment;

FIG. 2 is a perspective, exploded view of a fastener system for use with the spinal system of FIG. 1;

FIG. 3 is an exploded view of instrument for use with inserting and/or tensioning a cord within the spinal system of FIG. 1;

FIG. 4 is a close-up view of the cord attached to the threaded rod of the instrument in FIG. 3;

FIG. 5 is an assembled view of the instrument of FIG. 3;

FIG. 6 is a perspective view of the instrument of FIG. 3 with the compressor tube and shaft removed for clarity;

FIG. 7 is a compressed view of the instrument of FIG. 6 to tension the cord within the spinal system of FIG. 1;

FIG. 8 is an alternative version of an instrument suitable for tensioning the cord according to another embodiment;

FIG. 9 is another version of an instrument suitable for tensioning the cord according to another embodiment;

FIGS. 10-16 depict additional systems for tensioning the cord according to other embodiments;

FIGS. 17A-17C depict perspective and front views of a dual head fastener system according to one embodiment with two tulips each configured for receiving a cord and a fastener configured to anchor the fastener system to the vertebral body;

FIGS. 18A-18D show a dual head fastener system with two tulips and a fastener offset in either the cranial or caudal direction according to another embodiment;

FIGS. 19A-19C show a fastener system with a plate having a single tulip and a fastener having an integrated tulip positionable through an opening in the plate to secure two adjacent cords according to another embodiment;

FIGS. 20A-20D show a fastener system including a first tulip and a fastener with an integrated tulip according to another embodiment;

FIGS. 21A-21B show perspective and front views, respectively, of an integrated monoaxial fastener system to allow multiple cords to be fixated to the same fastener according to one embodiment;

FIG. 22 shows a front view of an integrated monoaxial fastener system with the threaded portion laterally offset to one side according to one embodiment;

FIGS. 23A-23B show a dual headed fastener system including a staple according to one embodiment;

FIGS. 24A-24B show a single head fastener system including an intermediate locking mechanism allowing multiple cords to be stacked on top of one another according to one embodiment;

FIG. 25 shows another embodiment of a single headed fastener with two cords configured to be stacked directly on top of one another;

FIGS. 26A-26B show perspective and front views, respectively, of a single head fastener system allowing for multiple cords to be placed laterally adjacent to one another and secured using a single locking mechanism according to one embodiment; and

FIGS. 27A-27B show perspective and front views, respectively, of a single head fastener system with adjacent cords configured to each be secured independently according to another embodiment.

### DETAILED DESCRIPTION

Embodiments of the disclosure are generally directed to scoliosis correction devices, systems, methods of use, and instruments for installing the same. The scoliosis correction system may be used to apply fixation on the convexity of the scoliotic vertebrae, for example. A rod or flexible cord(s), for example, may be used to limit growth on the convex side and allow unilateral growth on the concave side of the spine. In some embodiments, two or more cords may be used to provide additional rotational stability and/or improve long term dynamic performance.

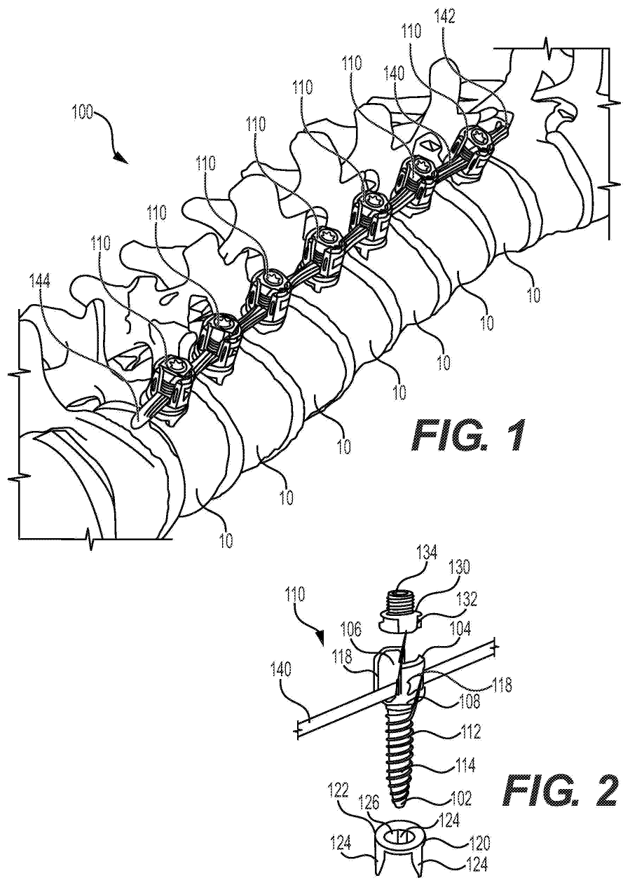

Referring now to FIG. 1, a scoliosis correction system 100 may include one or more fastener systems 110 secured to bone or vertebrae 10. The fastener systems 110 may be attached to an anterior aspect of each of the vertebral bodies 10, for example, adjacent to the rib head. Although seven fastener systems 110 are exemplified in the system 100 shown in FIG. 1, it will be appreciated that the surgeon could select any suitable number of fastener systems 110 as the surgeon deems appropriate to correct the deformity. The one or more fastener systems 110 are configured to retain a traditional rod or a cord 140 extending from a first end 142 to a second end 144. The rod or cord 140 is configured to be retained within the fastener systems 110 and tensioned to intraoperatively correct the deformity of the spine. The scoliosis correction system 100 may be implanted using an anterior or posterior spinal approach, for example. In particular, the system 100 may be implanted through a thoracoscopic approach or a mini-open thoractotomy to help minimize tissue disruption and/or scar tissue formation. The tensioned rod or cord 140 may provide for spinal alignment to maintain stability while allowing growth in skeletally immature patients.

The cord 140 may be a cable, wire, band, flexible or elastic member, for example. In one embodiment, the cord 140 is a narrow, flexible band. In some embodiments, the cord 140 is between 250-800 mm in length, for example, or may be cut to length, but any suitable length may be selected based on the deformity being corrected. As shown, the cord 140 is a single continuous cord extending from the first end 142 to the second 144, but it is also envisioned that more than one cord may be used or a section thereof may be coupled to another cord, rod, or other device, if desired. In some embodiments, the cord 140 is composed of a polymer, such as polyethylene terephthalate (PET), but any suitable biocompatible material may be selected. Preferably, the cord 140 is able to be placed under tension to achieve the proper amount of correction to the spine without fusion.

Turning to FIG. 2, the fastener system 110 may include a fastener 112, a staple 120, and a locking cap 130. The fastener 112 may extend from a distal end 102 to a proximal end 104. The distal end 102 may be configured to be received with the bone 10, and the proximal end 104 may be configured to sit above the bone 10. The fastener 112 may include a shaft portion or a threaded portion 114 and a yoke, tulip, or head portion 116. The threaded portion 114 may terminate at the distal end 102, for example, as a point, tapered to a narrowed end, or a blunt tip to facilitate insertion in the bone 10. In one embodiment, the distal end 102 may form a blunt tip for safe bicortical purchase in the bone 10. The threaded portion 114 may include any suitable type of threading. In one embodiment, the thread includes a double lead thread for rapid insertion, for example.

The yoke, tulip, or head portion 116 may be configured to move relative to the threaded portion 114. In one embodiment, the head portion 116 may be able to pivot about a longitudinal axis defined by the shaft portion of the fastener 112, thereby allowing for monoaxial movement of the head portion 116 relative to the threaded portion 114. In another embodiment, the head portion 116 may be able to move in a polyaxial manner relative to the threaded portion 114. It is also envisioned that the head portion 116 may be fixed relative to the threaded portion 114.

The head portion 116 of the fastener 112 may be in the form of a yoke or tulip defining a bore 106 therethrough, a body 108, and arms 118 that extend upwardly from the body 108. As shown in FIG. 2, the arms 118 may define a U-shaped channel sized to receive rod or cord 140. Each of the arms 118 has an interior surface configured to engage with the locking cap 130. Each of the arms 118 may also include an outer surface which defines a tool engagement groove for holding the head portion 116 with a suitable tool (not illustrated).

The locking cap 130 may be formed of two parts: an outer portion 132 and an inner portion 134. The outer portion 132 may define an opening therethrough for receiving the inner portion 132. The outer portion 132 may provide a non-threaded interface with the head portion 116 of the fastener 112. The inner portion 132 may be threaded to engage with interior threads within the outer portion 132. One or more features of the head portion 116 and locking cap 130 may be similar to those described in U.S. Pat. No. 8,888,827, which is incorporated by reference herein in its entirety for all purposes.

The fastener system 110 may further include a staple 120. The staple 120 may be formed of a ring 122 defining an opening 126. The ring 122 may be configured to surround the threaded portion 114 of the fastener 112 and a plurality of prongs 124 may extend from the ring 122 and are configured to engage the bone 10. In the embodiment shown, the ring 122 is a continuous ring, but it may also be split or otherwise shaped. In addition, the embodiment shown provides for three prongs 124 extending downwardly from the ring 122 and toward the distal end 102 of the fastener 112. The prongs 124 may help to increase the vertebral body fixation and/or increase resistance to screw toggle. Although three prongs 124 are exemplified, it will be appreciated that a different number or orientation of the prongs 124 may be selected.

The system 100 or components thereof may be comprised of any suitable biocompatible materials. For example, the staples 120, fasteners 112, and locking caps 130 may be manufactured from various biocompatible materials, such as metals, polymers, ceramics or combinations thereof. Exemplary materials include titanium (and titanium alloys), cobalt-chrome, stainless steel, and/or polyetheretherketone (PEEK), for example. In one embodiment, the fastener 112 (e.g., the threaded portion 114) is coated with a hydroxyapatite (HA) coating, which may help to promote bony ongrowth.

When installing the system 100, the one or more staples 120 may be inserted first or in tandem with the fastener 112. In one embodiment, the staples 120 may be positioned on the anterior aspect of the vertebral body 10. After the staple 120 is fully seated with a bottom surface of the ring 122 against an outer surface of the bone 10, the fastener 112 may be inserted through the opening 126 in the ring 122. The fastener 112 may be driven into the vertebral body 10 through the center of the staple 120. This process is repeated for all desired levels. Once the fastener systems 110 are placed, the cord 140 may be selected and cut to length. The cord 140 is positioned within the head portions 116 of each fastener 112. After the cord 140 is positioned within the head portion 116 of the fastener 112, the locking cap 130 may be positioned within the opening 106 such that the outer portion 132 is non-threadedly locked to the head portion 116 (e.g., rotated 90 degrees). This allows the cord 140 to be captured within the head portion 116 but still able to be tensioned. After final tensioning of the cord 140 is achieved, the inner portion 134 of the locking cap 130 may be rotated and threaded downwardly to contact and secure the cord 140 in its final position.

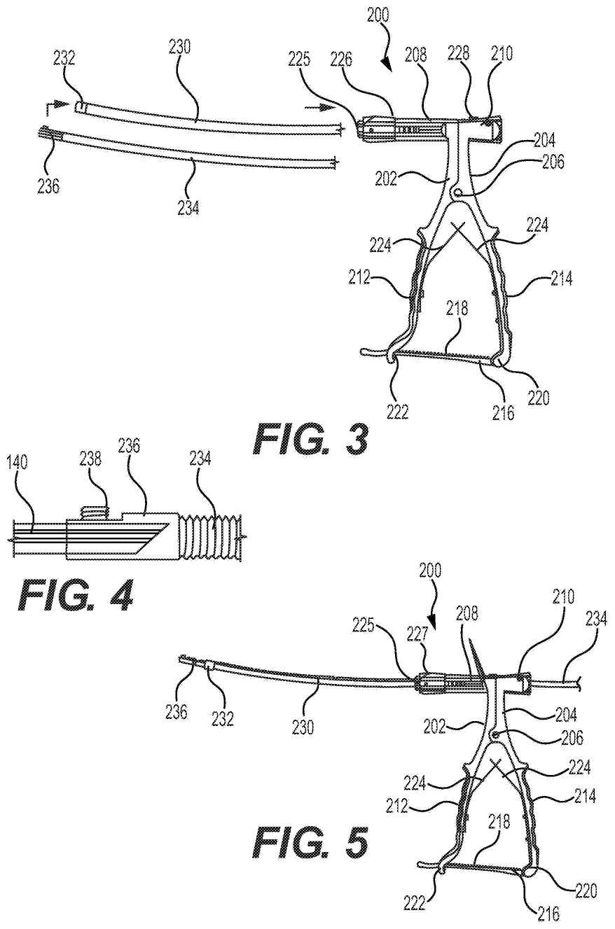

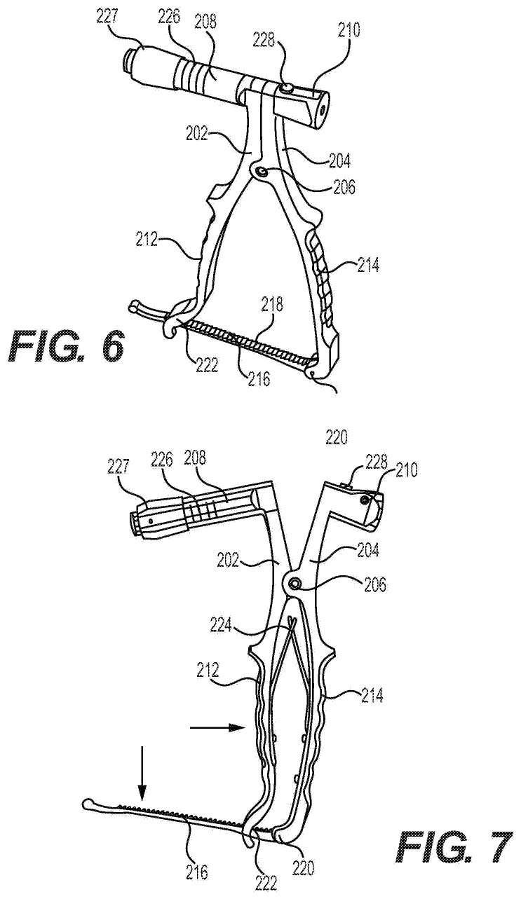

Turning now to FIGS. 3-6, an instrument 200 for tensioning a cord, for example, the cord 140 in system 100, according to one embodiment will be described. The instrument 200 may be configured to compress the spine during a thoracoscopic or open anterior spinal deformity correction procedure, for example. The instrument 200 may work with either a fusion or non-fusion procedure. It may be ineffective to compress the spine by manipulating minimally invasive surgical (MIS) tubes outside the wound because the tubes tend to flex. Instead, instrument 200 may be used in order to compress at the level of the respective fasteners 112. Furthermore, the instrument 200 is able to fit within a round, tubular port (e.g., 15 mm diameter) which is placed in the space between adjacent ribs. The instrument 200 can exert a suitable amount of compression on the spine, for example, approximately up to 440N [100 lbf] maximum.

The instrument 200 may include a first portion 202 and a second portion 204 coupled to the first portion 202 at a pivot point 206. The pivot point 206 may be provided by a pivot pin or other suitable mechanism to allow for pivotally connecting the first and second portions 202, 204 together. The first portion 202 includes a first tubular member 208 and the second portion 204 include a second tubular member 210. In a first position (e.g., shown in FIG. 3), the first and second tubular members 208, 210 are aligned along a common axis such that a central longitudinal opening extends therethrough. The first portion 202 defines a first handle portion 212 extending from the first tubular member 208 and the second portion 204 defines a second handle portion 214 extending from the second tubular member 210. In a second, compressed position (e.g., shown in FIG. 7), the first and second tubular members 208, 210 are not aligned along a common axis and are angled relative to one another.

The distal ends of the first and second handle portions 212, 214 may be connected with a ratchet 216. The ratchet may extend from the distal end of the second handle portion 214, for example, at a pivot 220, and engage with a pawl 222 on the first handle portion 212. When the first and second handle portions 212, 214 are squeezed towards one another, the pawl 222 can translate along teeth 218 on an upper surface of the ratchet 216 to maintain the first and second handle portions 212, 214 in the compressed configuration (e.g., shown in FIG. 7). The teeth 218 may be uni-directionally oriented to allow movement of the first handle 212 towards the second handle 214. The ratchet 216 may be disengaged from the pawl 222 to release the first handle portion 212 relative to the second handle portion 214. One or more springs 224 may be provided along inner surfaces of the handle portions 212, 214 to provide resistance to the instrument 200.

The first tubular member 208 may define a gauge sleeve 227 and a plurality of indicia 226. When the force gauge 227 moves, the plurality of indicia 226 may indicate the amount of compression force provided by the instrument 200. For example, the plurality of indicia 226 may be etchings provided, for example, in 100N increments. The second tubular member 208 may retain a button 228 configured to unlock the shaft 234 and allow the cord 140 to move within the instrument 200.

In operation, a sleeve or compressor tube 230 is inserted into opening 225 in the first tubular member 208. A cap 232 may be place on the end of the compressor tube 230 opposite the end that was inserted into the instrument 200. The cap 232 may be, for example, a PEEK cap that is firmly secured to the end of the compressor tube 230. Next, a shaft 234 may be inserted into the open end of the tube 230. The shaft 234 may be a threaded shaft 234 that is threaded along a portion or the entire length of the shaft 234. The shaft 234 may be a flexible threaded rod, for example, constructed of polyetheretherketone (PEEK), polyoxymethylene (POM), or a similar flexible plastic. The threaded shaft 234 may have a chuck or collet 236 at one end. When the shaft 234 is inserted in the compressor tube 230, the end of the threaded shaft 234 and/or the collet 236 may be exposed beyond the end of the compressor tube 230 (e.g., as shown in FIG. 5). The button 228 may be depressed to slide the threaded shaft 234 through the first and second tubular members 208, 210.

As best seen in FIG. 4, the cord 140 may be inserted into an opening in the collet 236. The cord 140 may be secured to the collet 236, for example, with a fastener 238. The fastener 238 may be a threaded set screw or any other suitable mechanism for securing the cord 140 to the shaft 234. In the alternative, the collet 236 with the cord 140 previously connected thereto may be threaded onto the threaded shaft 234 to thereby secure the cord 140 to shaft 234.

The instrument 200 is operated by exerting a pull on the cord 140 relative to the sheath or tube 230 with the hand lever, as a result, the calipers or handles 212, 214 are compressed together. The rod or cord 140 may be attached to the flexible threaded rod 234 using the collet 236. The collet 236 may automatically grab the rod or cord 140 when pulled into the tube 230. It may be advantageous that both the flexible threaded rod 234 and tube 230 are pliable, thereby making it easier for the surgeon to guide the rod or cord 140 to a fastener 110 within the pleural space.

According to one embodiment, the procedure may follow the following sequence: (1) the patient may be placed in the lateral decubitus position with the convexity of the scoliotic curve facing up; (2) monoaxial pedicle screws 110 may be placed across the appropriate vertebral bodies, either through ports or through an open surgical exposure; (3) the compressor instrument 200 is assembled by pushing compressor tube 230 into the end and sliding flexible threaded rod 234 into the tube 230; (4) the rod (for fusion surgery) or cord 140 (for non-fusion surgery) is attached to collet 236 and pulled into the tube 230; (5) the tube 230 is inserted into the pleural space through either an incision or a port placed into an incision, which is placed caudally to the most caudal fastener 110; (6) the most cranial fastener 110 is located and the rod or cord 140 is placed into the yoke or head 116 of that screw and captured with a counter-torque; and the locking cap 130 of the fastener 1120 is tightened on the rod or cord 140; (7) the tube 230 is pulled back to expose more of the rod or cord 140, which is then placed into the yoke or head 116 of the adjacent (caudal) fastener 1120 and captured with the counter-torque; (8) the tube 230 is pushed forward until it abuts the countertorque; the compressor handles 212, 214 are squeezed until the desired compression force is achieved on the spine; and the locking cap 130 is fully tightened by threading the inner portion 134 of the locking cap 130 onto the cord 140; (9) steps 7 and 8 are repeated for the remainder of the screws 110; and (10) in the case where a rod has been implanted, the collet 236 is loosened and the compressor instrument 200 is removed from the pleural space; or in the case where a cord 140 has been implanted, the cord 140 may be cut with an electrocautery device, scalpel, scissors, or the like; and the compressor 200 is removed from the pleural space.

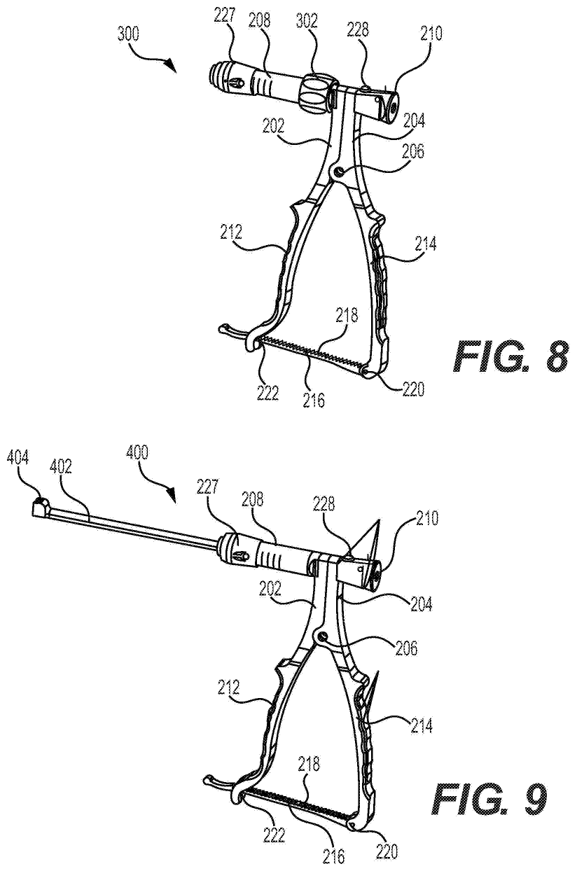

Turning now to FIG. 8, an alternative compression instrument 300, similar to instrument 200 will be described. Instrument 300 is similar in function to instrument 200 and like elements will be labeled with the same reference numbers. In addition to the features of instrument 200, instrument 300 provides a secondary method of applying compression to the construct. In this embodiment, the first and second tubular members 208, 210 may be additionally expanded apart by threads or another similar mechanism. The expansion of this fine-tuning mechanism increases the distance between the lock mechanism and the sheath or tube 230, applying force to the flexible rod 234, which holds the rod or cord 140. In particular, a thumb wheel 302 with threads configured to engage the threads of the threaded shaft 234 may provide finer control of the amount of compression applied to the rod or cord 140. In conjunction with the squeeze handles 212, 214, instrument 300 enables two stages of compression for both increased force application and finer control of applied force. In other words, handles 212, 214 provide for large amounts of compression and thumb wheel 302 provides for small amounts of compression, thereby allowing the surgeon to pinpoint a precise amount of compression on the rod or cord 140.

Turning now to FIG. 9, an alternative compression instrument 400, similar to instrument 200 will be described. Instrument 400 is similar in function to instrument 200 and like elements will be labeled with the same reference numbers. In instrument 400, the flexible tube 230 has been replaced with a rigid shaft 402. The rigid shaft 402 interfaces with the force gauge 227 at the proximal end. The rigid shaft 402 has a smooth channel or cannula which guides the rod or cord 140 and the flexible shaft 234 therein. At the distal tip 404 of the shaft 402, the channel may be curved, for example, to redirect the path of the captured rod or cord 140, allowing compression to be applied at an angle or perpendicular to the axial force on the construct.

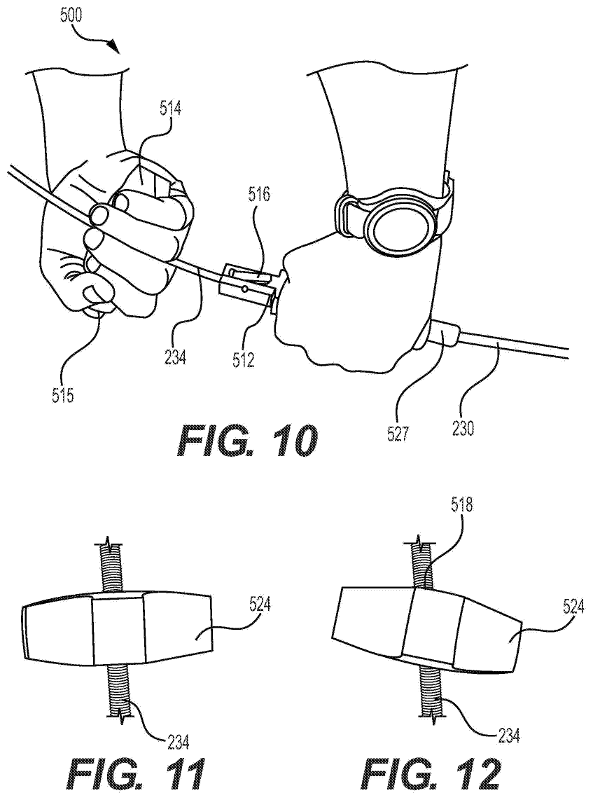

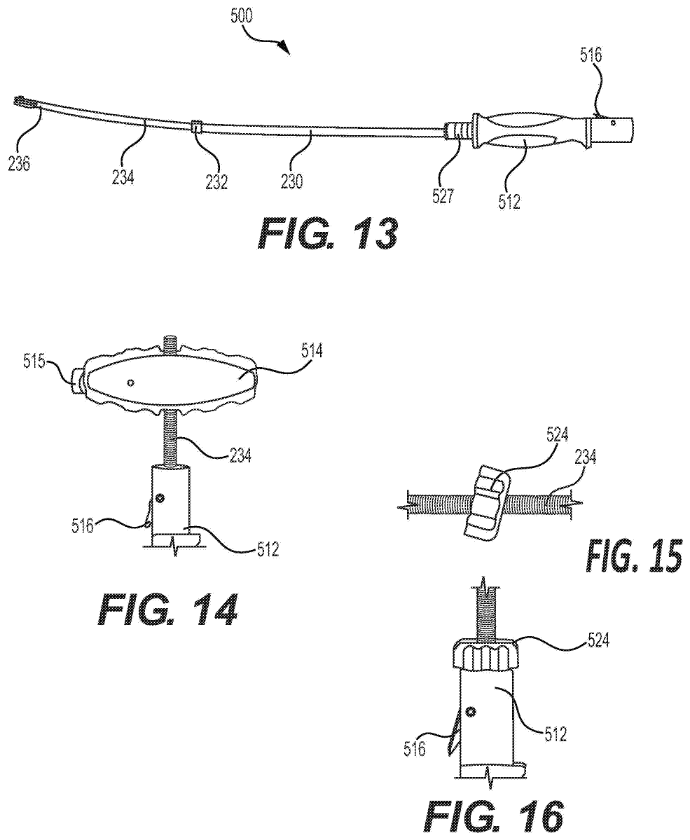

Turning now to FIGS. 10-16, alternative compression instruments 500 are shown. The compressor 500 may function in a manner similar to instrument 200, however, instead of squeeze handles 212, 214, the surgeon may hold two separate handles 512, 514 (one in each hand) and pulls them apart from one another to impart the compressive forces on the rod or cord 140. The main handle 512 grasps the compressor tube 230 and includes a ratcheting mechanism 516 and a force gauge 527. The other handle 514, may be in the form of a T-handle, for example, and may have a release button 515 so that it can be easily moved to a comfortable position on the flexible threaded shaft 234. This embodiment may be a good option for applying small compression forces to the spine. As an alternative to the T-handle 514, a knob 524 may be used. The knob 524 may have an internal thread and works like a nut when threaded onto the flexible shaft 234. To facilitate quick movement of the knob 524 along the threaded shaft 234, an off-axis through hole 518 may be drilled through the center of the knob 524, for example. When the knob 524 is tilted (canted), for example, as shown in FIGS. 12 and 15, then the internal threads are disengaged and the knob 524 can be rapidly pushed or pulled along the flexible shaft 234. For example, the knob 524 may be moved along shaft 234 until it abuts the main handle 512, for example, as shown in FIG. 16. Once the knob 524 contacts the main handle 512, the knob 524 can be re-aligned to be coaxial with the flexible shaft 234 and turned to apply compressive force to the spine. The inline handle 514 or knob 524 may tension the cord 140 between the final tightened screw and the most caudal screw. The compressive force, as indicated by the gauge 527 may be up to 150N of compressive force (e.g., indicated in 50N increments).

The embodiments described herein can provide large compressive, corrective forces and/or small fine-tuned corrective forces to be placed on the spine, for example, in a minimally invasive surgery. The forces may be exerted directly on the fasteners as opposed to indirectly exerting force on a long, flexible instrument attached to the screws. This allows the surgeon to control the force placed on the spine by offering force feedback to the surgeon in the form of the force gauge. The allows simplified placement of the cord into the yoke or head portion of an adjacent pedicle screw by simply pivoting the instrument relative to an already tightened pedicle screw. These systems can facilitate scoliosis correction and serve as a means for deformity correction with or without fusion.

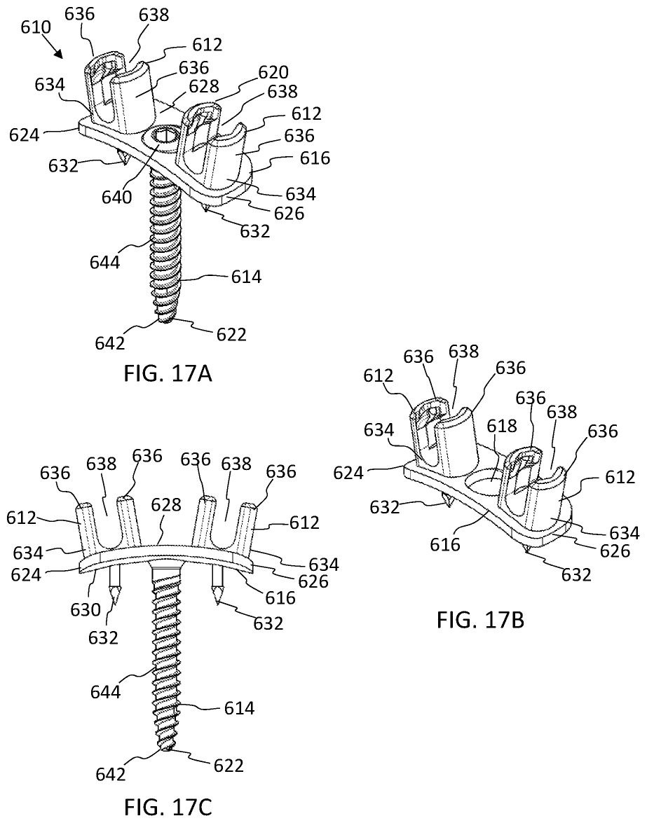

Turning now to FIGS. 17-27, the scoliosis correction system 100 may include two or more cords 140, which may provide additional rotational stability and/or improve long term dynamic performance. Fusionless anterior scoliosis correction may be of interest for surgeons who treat adolescent idiopathic deformity. In a vertebral body tethering (VBT) or fusionless anterior scoliosis correction (ASC) procedure, a screw is placed in each vertebral body along the scoliotic curve, and a flexible cord or tether is used to provide corrective forces. In some cases, this technique may be insufficient compared to a traditional fusion-based rod construct when rotational correction is limited due to the flexible, non-rigid nature of the construct.

In some cases, it may be helpful to provide a double cord construct, in which two screws are placed in each vertebral body and two cords/tethers are installed to allow for additional rotational stability. Not all vertebral bodies are able to accommodate two vertebral body screws, however. For example, vertebral bodies, particularly in upper thoracic, may not be large enough to provide for adequate fixation. In other cases, particularly in the case of a double major curve, the technique requires the transitional vertebral bodies be fixated from either lateral side for stability, which currently only allows for one vertebral body screw on each side, and poses a challenge in terminating a double cord construct.

Embodiments shown in FIGS. 17-27 are configured to retain two or more cords 140 with a single bone fastener, thereby providing more robust fixation systems and methods for scoliosis correction. The following embodiments provide additional surgical options for creating double cord constructs in VBT or ASC techniques. The embodiments described herein facilitate dual cord constructs while reducing the number of vertebral body screws required. This may help surgeons to create more stable constructs, which may result in better correction of the patients' deformities. Although the embodiments are described with two cords 140, it will be appreciated that the devices may also be used with two fixation rods or one rod and one cord combination, for example.

With emphasis on FIGS. 17A-17C, a dual headed fastener system 610 may include a pair of cord anchors, head portions, or tulips 612 each configured to retain a cord 140 and a bone fastener 614 configured to secure the system 610 to bone. The tulips 612 may be affixed to a base or plate 616 and the bone fastener 614 may be receivable in an opening 618 through the plate 616. The system 610 may include a proximal end 620, a distal end 622, a first lateral side 624, and a second lateral side 626.

The base or plate 616 may have an upper surface 628 and an opposed lower surface 630. The plate 616 may extend between the first and second laterals sides 624, 646. The tulips 612 may be separated a distance from one another and extend upwardly from the upper surface 618 of the plate 616. The two tulips 612 may be laterally offset from one another and integrated into the same plate 616. It is also envisioned that the tulips 612 may be moveable or otherwise secured to the plate 616 using set screws, adhesive, or other suitable means. The plate 616 may be curved, bowed, or contoured, for example, to mimic the bone surface. The upper surface 618 may have a generally convex surface and the lower surface 630 may have a generally concave surface. The outer-most lateral sides 624, 626 of the plate 616 may be curved downwardly toward the distal end 622 of the system 610. In this manner, the tulips 612 may be angled outwardly and away from one another to receive the respective cords 140.

One or more stabilizing spikes, anchors, or prongs 632 configured to engage bone may extend downwardly from the lower surface 630 of the plate 616. Each prong 632 may have an elongated body, for example, forming a cylindrical tine with a pointed or sharpened distal-most end. The sharpened end of the prong 632 may form a conical tip, for example. In the embodiment shown, two prongs 632 extend downwardly below each tulip 612 and toward the distal end 622 of the fastener 614. The pair of prongs 632 may be aligned in parallel to one another. The prongs 632 may help to increase the vertebral body fixation and/or to reduce rotation of the construct. Although two prongs 632 are exemplified, it will be appreciated that a different number, location, or orientation of the prongs 632 may be selected.

First and second tulips 612 may be integral with the upper surface 628 of the plate 616. Each tulip 612 may be in the form of a head or yoke defining a body 634 with arms 636 that extend upwardly from the body 634. Each pair of arms 636 may extend generally in parallel to one another. The arms 636 may define a U-shaped channel 638 sized to receive cord 140 from the proximal end 620 of the system 610. Each of the arms 636 has an interior surface configured to engage with a locking cap (such as locking cap 130, 728, 768) to secure the cord 140 in the tulip 612.

The bone fastener 614 is receivable through opening 618 in the plate 616, thereby securing the plate 616 to bone. The fastener 614 may extend from a proximal head 640 to a distal end 642. The distal end 642 may be configured to be received with the bone 10, such as a vertebral body, and the proximal head 640 may be configured to sit within the opening 618 in the plate 616. The shape of the proximal head 640 and opening 618 may be provided to allow for monoaxial, uniaxial, or polyaxial movement of the fastener 614 relative to the plate 616. The single bone fastener 614 is configured to secure the plate 616 to the bone.

The fastener 614 may include a shaft portion or a threaded portion 644, which may terminate at the distal end 642, for example, as a point, tapered to a narrowed end, or a blunt tip to facilitate insertion in the bone 10. In one embodiment, the distal end 642 may form a blunt tip for safe bicortical purchase in the bone 10. The threaded portion 644 may include any suitable type of threading. The opening 618 and bone fastener 614 may be located centrally between the tulips 612 or at either end of the plate 616. The additional anchor point for the bone fastener 614 may be located along the same lateral offset as the tulips 612. In this manner, a single bone fastener 614 may be used to secure two cords 140 to correct the patient deformities.

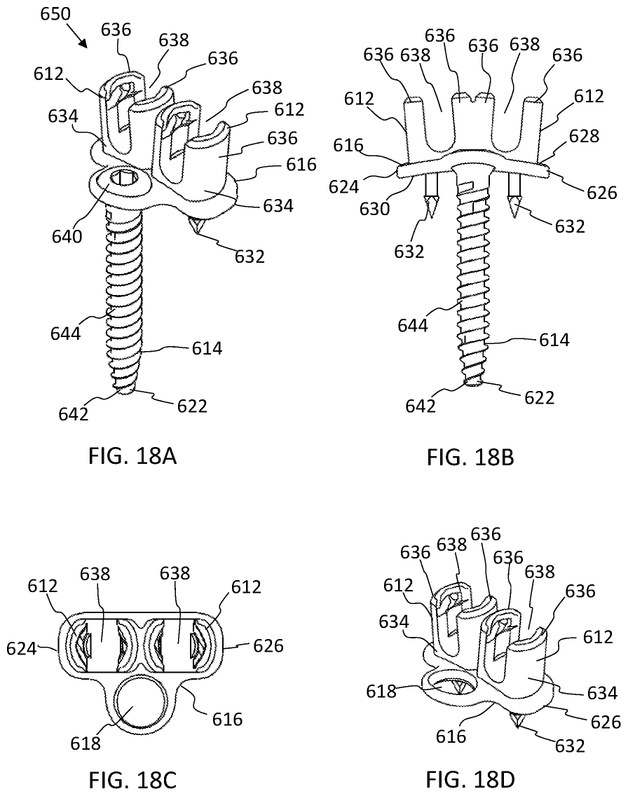

Turning now to FIGS. 18A-18D, a dual headed fastener system 650, similar to fastener system 610, is shown, and like elements are labeled with the same reference numbers. In this embodiment, the tulips 612 laterally offset from each other, are positioned closer together and the opening 618 and fastener 614 is positioned cranially or caudally. The inner arms 636 of the tulips 612 may be joined together or integrally formed at the center of the device. As best seen in the top view of FIG. 18C, the two tulips 612 for the cords 140 are integrated together on the plate 616, with an anchor point for the vertebral body screw 614 offset in either a cranial or caudal direction. The plate 616 is widened to accommodate the opening 618 for the fastener 614. The opening 618 and fastener 614 may be centrally located to the plate 616, as shown in the front view of FIG. 18B, or may be offset laterally to one side. The plate 616 may include one or more stabilizing spikes, anchors, or prongs 632 to help reduce rotation of the construct.

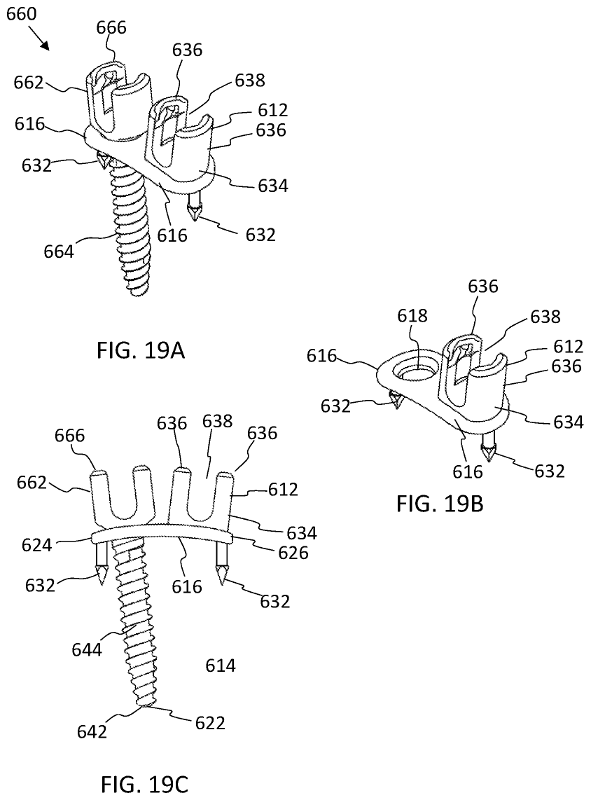

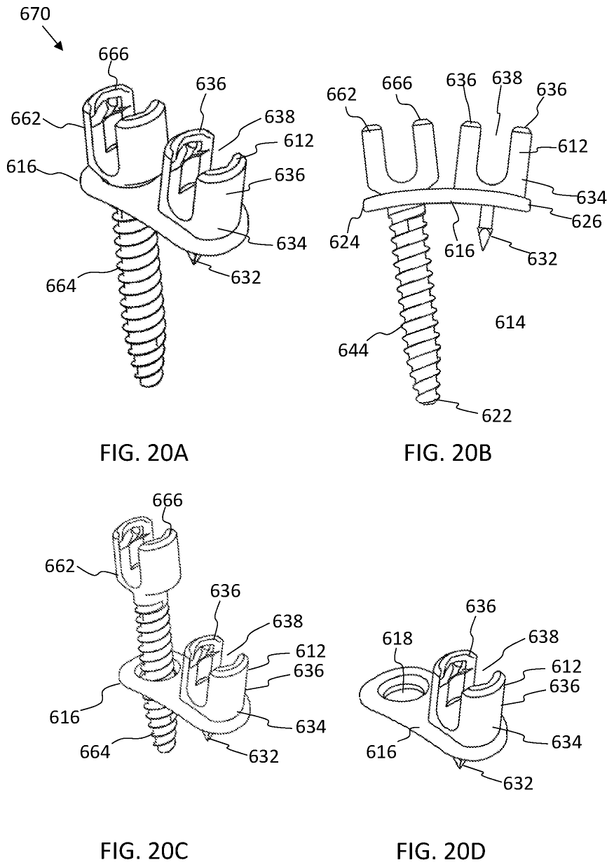

Turning now to FIGS. 19A-19C and 20A-20D, fastener systems 660, 670 are shown. In these embodiments, the fastener system 660, 670 includes a single integrated tulip 612 affixed to plate 616 and a separate bone fastener 662 with an integrated anchor tulip 666. Similar to bone fastener 112, fastener 662 may include a shaft portion or threaded portion 664 and a yoke, head, or tulip 666 configured to receive one cord 140.

As best seen in FIGS. 19B and 20D, the plate 616 includes an opening 618 laterally offset to the integral tulip 612. A first cord 140 is configured to be positioned and retained in integral tulip 612. The vertebral body screw 662 is passable through the opening 618 in plate 616 and integrated anchor tulip 666 is configured to retain the second cord 140. The fastener 662 may be permitted to angulate in the opening 618 and relative to the plate 616 during insertion. The plate 616 may optionally include one or more stabilizing spikes, anchors, or prongs 632 to reduce rotational movement of the construct. In the embodiment shown in FIGS. 19A-19C, two parallel prongs 632 are provided on the lateral ends 624, 626 of the plate 616. The fastener system 670 shown in FIGS. 20A-20D is similar to fastener system 660 except a single prong 632 is provided beneath the integral tulip 612. It is also envisioned that a second smaller vertebral body screw could also be used to stabilize the construct.

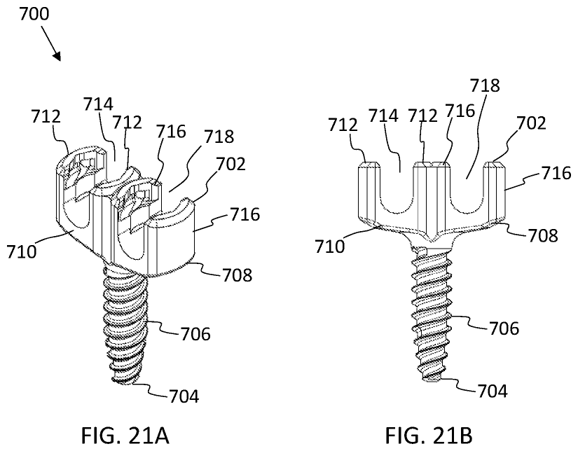

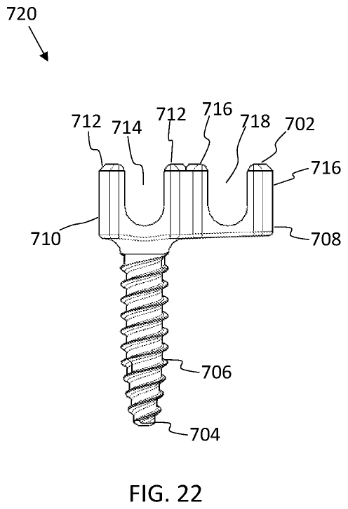

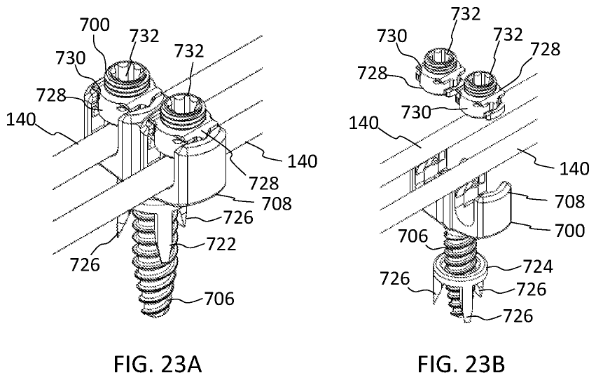

Turning now to FIGS. 21A-21B, FIG. 22, and FIGS. 23A-23B, dual headed monoaxial fasteners 700, 720 are shown. The dual headed fasteners 700 allow for multiple cords 140 to be captured side by side in a single device. In this manner, multiple cord fixation points allow multiple cords 140 to be fixated to the same vertebral body screw. The threaded portion 706 of the device body may be centered on the device 700 (as shown in FIGS. 21A-21B) or offset to one side of the device 720 (as shown in FIG. 22). The dual headed screw device 700 may be used with a staple (as shown in FIGS. 23A-23B) for stability, similar to system 110 used in single cord non-fusion constructs or anterior fusion procedures.

With emphasis on FIGS. 21A-21B, the dual headed monoaxial fastener system 700 extends from proximal end 702 to a distal end 704. The distal end 704 may be configured to be received in the bone 10, and the proximal end 702 may be configured to sit above the bone 10. The fastener 700 may include a shaft portion or a threaded portion 706 and a double headed yoke or tulip 708. The threaded portion 706 may terminate at the distal end 704, for example, as a point, tapered to a narrowed end, or a blunt tip to facilitate insertion in the bone 10. In one embodiment, the distal end 704 may form a blunt tip for safe bicortical purchase in the bone 10. The threaded portion 706 may include any suitable type of threading for bone purchase.

The dual headed yoke or tulip 708 may be integral with the shaft portion 706 or may be configured to move relative to the shaft portion 706. The double tulip 708 of the fastener 700 may include a body 710 with a first pair of arms 712 that extend upwardly from the body 710 to define a first U-shaped channel 714 sized to receive first cord 140 and a second pair of arms 716 that extend upwardly from the body 710 to define a second U-shaped channel 718 sized to receive second cord 140. The inner arms 712, 716 may be in contact or integrated in the center of the tulip 708 to form a unitary body. The first and second channels 714, 718 may be aligned in parallel or another suitable configuration to retain the cords 140. Each pair of arms 712, 716 has an interior surface configured to engage with a locking cap (such as locking cap 130, 728, 768), which secures the cord 140 in the respective channel 714, 718. One or more arms 712, 716 of the double tulip 708 may also include an outer surface which defines a tool engagement groove for holding the double tulip 708 with a suitable tool (not illustrated).

As shown in the embodiment in FIGS. 21A-21B, the threaded shaft 706 may be centrally located relative to the double tulip 708. The threaded shaft 706 may extend beneath the double tulip 708 along a central longitudinal axis of the device 700. In this manner, the shaft 706 is centered between the first and second channels 714, 718. Alternatively, as shown in the embodiment in FIG. 22, the threaded shaft 706 may be offset to one side of the double tulip 708. For example, the threaded shaft 706 may be generally aligned with one channel 714, 718. Although shown aligned with first channel 714, it will be appreciated that the shaft 706 may be aligned with second channel 718 or otherwise offset along the body 710 of the tulip 708.

With emphasis on FIGS. 23A-23B, the dual headed monoaxial fastener system 700 may further comprise a staple 722 for added stability. Similar to staple 120, staple 722 may include a ring 724 defining an opening, which is configured to surround the threaded portion 706 of the fastener 700 and a plurality of prongs 726 extending downwardly from the ring 724 configured to engage bone. In the embodiment shown, the ring 724 is a continuous ring, but it may also be split or otherwise shaped. The embodiment shown provides for three prongs 726 extending downwardly from the ring 724 and toward the distal end 704 of the fastener 700. The prongs 726 may be located equidistantly about the perimeter of the ring 724. Although three prongs 726 are exemplified, it will be appreciated that a different number or orientation of the prongs 726 may be selected. The prongs 726 may help to increase the vertebral body fixation and/or increase resistance to screw toggle.

Each of the cords 140 may be secured in the respective channels 714, 718 with a locking cap 728. Similar to locking cap 130, locking cap 728 may be formed of two parts: an outer portion 730 and an inner portion 732. The outer portion 730 may define an opening therethrough for receiving the inner portion 732. The outer portion 730 may provide a non-threaded interface with the arms 712, 714 of the double headed tulip 708. The inner portion 732 may be threaded to engage with interior threads within the outer portion 730. The inner portion 732 may define a recess configured to be engaged with a driver instrument, for example. One or more features of the interface and locking cap 728 may be similar to those described in U.S. Pat. No. 8,888,827, which is incorporated by reference herein in its entirety for all purposes. It will be appreciated that other suitable locking caps or securing members may be selected to secure the cords 140 in position. In this manner, the first locking cap 728 secures the first cord 140 in the first channel 714 and the second locking cap 728 secure the second cord 140 in the second channel 718, thereby allowing for the cords 140 to be positioned alongside one another. These cords 140 may be aligned in parallel or may be angled toward or away from one another depending on the channel 714, 718 configuration.

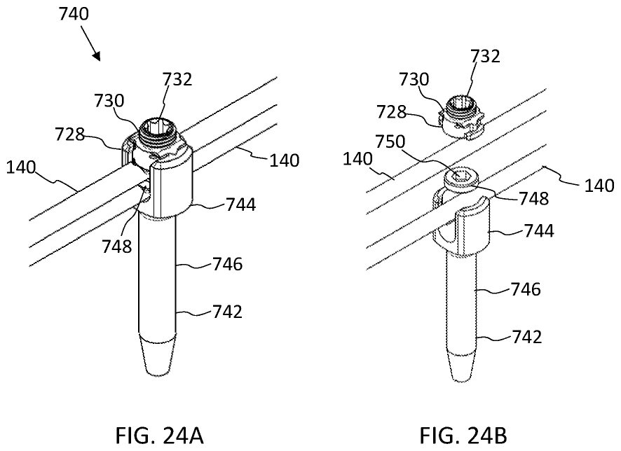

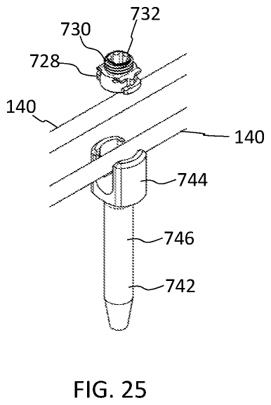

Turning now to FIGS. 24A-24B and 25, a single head fastener system 740 for securing more than one cord 140 is shown. In these embodiments, multiple cords 140 may be captured in a single tulip via a single locking mechanism. The cords 140 may be vertically stacked or layered on top of each other and in direct contact (as shown in FIG. 25) or with the option of an intermediate locking washer 748 to secure the first cord 140 prior to insertion of additional cords (as shown in FIGS. 24A-24B).

Similar to fastener system 110, fastener system 740 includes a fastener 742 with a tulip head 744 for retaining cords 140 and a locking cap 728 for securing the cords 140 therein. The fastener 742 may include a shaft portion or a threaded portion 746 extending from the yoke, head, or tulip portion 744. The threaded portion 746 is drawn without any threading on the screw body for clarity only. The tulip 744 may include a pair of arms defining a U-shaped channel sized to receive multiple cords 140 stacked on top of one another. An inner surface of the arms is configured to engage with the locking cap 728, thereby securing the stacked cords 140 inside the tulip 744.

In FIGS. 24A and 24B, the cords 140 are separated by a locking washer 748. The locking washer 748 may have a ring-like or disc-shaped body with a central opening or recess 750. The central recess 750 may be polygonal, such as hexagonal, or otherwise configured to be engaged with a driver or instrument. The locking washer 748 may have a diameter smaller than the inner distance between the arms of the tulip 744. The washer 748 may be configured to be rest in a channel, groove, or otherwise lock into the arms of the tulip 744 to thereby secure the lowermost cord 140 in position. After the locking washer 748 is positioned in the tulip 744, the upper cord 140 may be positioned on top of the locking washer 748. The locking washer 748 has a thickness configured to separate the first and second cords 140 such that the stacked cords 140 are not in contact with one another. The cords 140 may be arranged in parallel separated a distance determined by the thickness of the washer 748 along the longitudinal axis of the fastener 742. The stacked cords 140 may then be locked in position with a single locking cap 728.

Alternatively, as shown in FIG. 25, the cords 140 may be stacked in direct contact with one another. In this embodiment, the locking washer 748 is omitted and the cords 140 are stacked directly one on top of the other. The cords 140 may be arranged in parallel but in direct contact along their lengths. Both of the stacked cords 140 are then secured in position with a single locking cap 728.

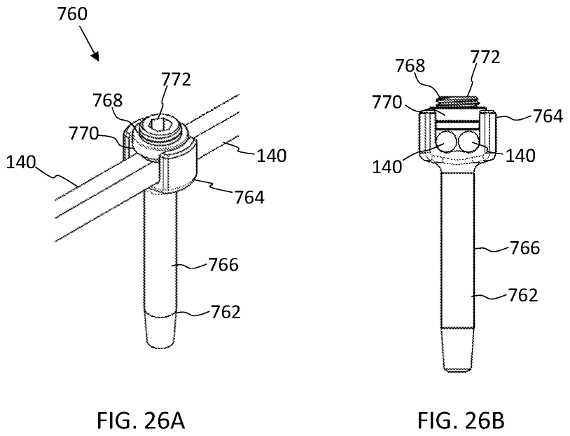

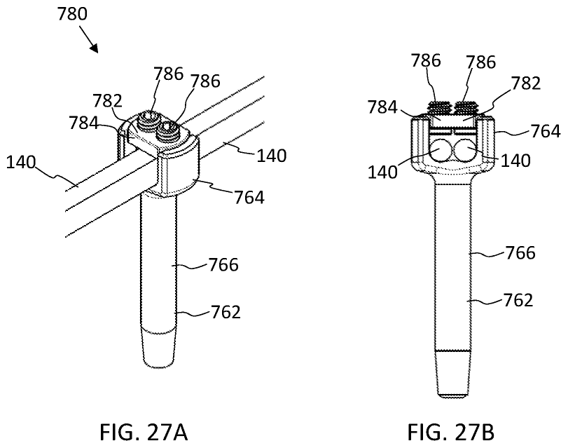

Turning now to FIGS. 26A-26B and 27A-27B, fastener systems 760, 780 for securing multiple cords 140 placed laterally adjacent to each other and secured using a single locking mechanism 768, 782 are shown. In these embodiments, the cords 140 are positioned in a single tulip 764 but are arranged laterally side by side. Similar to fastener system 740, fastener systems 760, 780 include a fastener 762 with a tulip head 764 for retaining the cords 140. The fastener 762 may include a shaft portion or a threaded portion 766 extending from the yoke, head, or tulip portion 764. The threaded portion 766 is drawn without any threading on the screw body for clarity only. The tulip 764 may include a pair of arms defining a U-shaped channel sized to receive the multiple cords 140 next to one another. In this manner, the U-shaped channel is sized wide enough to accommodate the thicknesses or widths of the multiple cords 140 arranged laterally adjacent to one another.

The arms of the tulip 764 are configured to engage with a locking cap 768, 782 thereby securing the cords 140 in the tulip 764. In the embodiment shown in FIGS. 26A-26B, locking cap 768 may be similar to locking cap 730 with an outer portion 770 and a single inner portion 772. The outer portion 770 may define a single opening therethrough for receiving the inner portion 772. The outer portion 770 may provide a non-threaded interface with the arms of the tulip 764. For example, the outer portion 770 may be positioned in a groove or channel along an internal surface of the arms of the tulip 708. The inner portion 772 may be threadedly engaged with interior threads within the outer portion 770. The inner portion 772 may define a recess configured to be engaged with a driver instrument, for example. A bottom surface of the inner portion 772 and/or outer portion 770 may contact the tops of the cords 140 to retain them in the tulip 764. Alternatively, a separate clamp, washer, or the like may be positioned between the bottom of the locking cap assembly 768 and the top of the cords 140, thereby securing the cords 140 in position when the inner portion 772 is threaded downwardly.

In the embodiment shown in FIGS. 27A-27B, the locking cap 782 includes two inner members 782 configured to secure each cord 140 independently. Locking cap 782 includes an outer member 784 with an elongate body defining two openings configured to receive two threaded inner members 782. The outer member 784 may be non-threaded and configured to be received in a recess or groove inside the arms of the tulip 764. As the threaded inner members 782 are threaded downwardly, a bottom surface of the inner members 782 may contact an upper portion of the respective cords 140 thereby securing the cords 140 in the tulip 764 independently. Although nonthreaded locking caps 768, 782 are exemplified to secure the flexible tethering cords 140, threaded engagement mechanisms, such as set screws, or other fixation methods could also be used.

The embodiments described herein are able to retain at least two flexible cords with a single bone fastener, thereby providing more robust fixation systems and methods for scoliosis correction. The dual cord constructs may help surgeons to create more stable constructs, which may result in better correction of the patients' deformities. The double cord constructs create additional surgical options in VBT and ASC procedures.

Although the invention has been described in detail and with reference to specific embodiments, it will be apparent to one skilled in the art that various changes and modifications can be made without departing from the spirit and scope of the invention. Thus, it is intended that the invention covers the modifications and variations of this invention provided they come within the scope of the appended claims and their equivalents. It is expressly intended, for example, that all ranges broadly recited in this document include within their scope all narrower ranges which fall within the broader ranges. It is also intended that the components of the various devices disclosed above may be combined or modified in any suitable configuration.

What is claimed is:

1. A fixation system for correcting scoliosis comprising:
first and second cords;
a base;
a tulip structure disposed on the base for receiving at least one of the first and second cords; and
a locking cap disposed in the tulip over at least one of the first and second cords and adapted to lock at least one of the first and second cords.

2. The fixation system of claim 1, wherein the tulip structure includes a body with a pair of upwardly extending arms, wherein the arms define a U-shaped channel therebetween sized and shaped to receive at least one of the first and second cords.

3. The fixation system of claim 1, wherein the base includes a plate having an upper surface and an opposed lower surface, wherein the tulip structure is integrated with the upper surface of the plate.

4. The fixation system of claim 3, wherein the plate is curved with the lower surface having a concave surface.

5. The fixation system of claim 3, wherein the plate includes one or more prongs extending downwardly from the lower surface of the plate.

6. The fixation system of claim 3 further comprising a second tulip structure integral with the upper surface of the plate, wherein each tulip structure retains one of the first and second cords.

7. The fixation system of claim 3, wherein the plate defines an opening extending therethrough, and a fastener is receivable through the opening in the plate.

8. The fixation system of claim 7, wherein the fastener includes a head and a threaded shaft portion configured to engage bone, wherein the head is receivable in the opening through the plate.

9. The fixation system of claim 1, wherein the locking cap includes a non-threaded outer portion and a threaded inner portion receivable through the outer portion, wherein threading the inner portion downwardly secures at least one of the first and second cords in the tulip structure.

10. A fixation system for correcting scoliosis comprising:
a single bone fastener including a threaded shaft portion configured to engage bone;
first and second tulips each including a body with a pair of upwardly extending arms defining a U-shaped channel therebetween; and
a plurality of cords configured to be secured in the first and second tulips, wherein the system secures the plurality of cords to the single bone fastener.

11. The fixation system of claim 10 further comprising first and second locking caps disposed in the first and second tulips over the cords and adapted to lock the cords therein.

12. The fixation system of claim 10, wherein both of the first and second tulips are affixed to a plate.

13. The fixation system of claim 12, wherein the fastener is receivable through a central opening located between the first and second tulips.

14. The fixation system of claim 12, wherein the plate includes a pair of prongs extending downwardly, wherein the prongs are positioned beneath each of the first and second tulips.

15. The fixation system of claim 10, wherein one of the first and second tulips is attached to a plate, and the other of the first and second tulips is integral with the bone fastener.

16. The fixation system of claim 15, wherein the fastener is receivable through a laterally offset opening through the plate, and the tulip attached to the bone fastener is positionable above the plate.

17. A method of correcting scoliosis of the spine comprising:
positioning a plate against a vertebra, the plate including a first tulip disposed on the plate for receiving a first cord and a second tulip disposed on the plate for receiving a second cord;
placing a single fastener through an opening in the plate and securing the fastener to the vertebra;
positioning a first cord through the first tulip and securing the first cord therein with a first locking cap; and
positioning a second cord through the second tulip and securing the second cord therein with a second locking cap, wherein the first and second cords are affixed to additional fastener systems on adjacent vertebrae to thereby stabilize the spine.

18. The method of claim 17, wherein the first and second cords are aligned in parallel.

19. The method of claim 17, wherein the first and second cords are tensioned to correct a curve of the spine.

20. The method of claim 17, wherein the plate includes one or more prongs extending downwardly from the plate configured to engage the vertebra and reduce rotation of the plate.
